(19) United States
(12) Patent Application Publication
(10) Pub. No.: US 2026/0309663 A1
(43) Pub. Date: Oct. 8, 2026

(54) SENSOR MODULE AND METHOD FOR MANUFACTURING A SENSOR MODULE

(71) Applicant: Sciosense B.V., AE Eindhoven (NL)

(72) Inventors: Ching-Yu Yuan, Ae Eindhoven (NL); Frederik Willem Maurits Vanhelmont, Ae Eindhoven (NL); Casper Van Der Avoort, Ae Eindhoven (NL)

(21) Appl. No.: 19/481,744

(22) PCT Filed: Mar. 21, 2024
(86) PCT No.: PCT/EP2024/057592

§ 371 Date: Nov. 5, 2025

(30) Foreign Application Priority Data
May 9, 2023 (DE) 102023112141.3
Jul. 27, 2023 (DE) 102023119941.2

(51) Int. Cl. G01N 27/12

(52) U.S. Cl. CPC G01N 27/121

(57) ABSTRACT

In an embodiment a sensor module includes a connection carrier, a sensor arranged on the connection carrier, a sensor cap surrounding the connection carrier and/or the sensor at least partially, an insert surrounding the sensor, the insert leaving an access opening of the sensor freely accessible, wherein the insert includes an elastic material.

### CROSS-REFERENCE TO RELATED APPLICATIONS

This patent application is a national phase filing under section 371 of PCT/EP2024/057592, filed Mar. 21, 2024, which claims the priority of German patent application no. 102023112141.3, filed May 9, 2023, and claims the priority of German patent application no. 102023119941.2, filed Jul. 27, 2023, each of which is incorporated herein by reference in its entirety.

### TECHNICAL FIELD

A sensor module and a method for manufacturing a sensor module are provided.

### SUMMARY

Embodiments provide a sensor module. Further embodiments provide a method for manufacturing a sensor module.

According to an embodiment, the sensor module comprises a connection carrier. For example, the connection carrier is a printed circuit board (PCB). Particularly, the connection carrier comprises electrical connection points and/or conductor tracts.

According to an embodiment, the sensor module comprises a sensor arranged on the connection carrier. Particularly, leads of the sensor are electrically conductively and/or mechanically stably connected to the electrical connection points, for example by soldering or gluing.

For example, the sensor is configured to detect a pressure, a temperature and/or a fluid. Particularly, the sensor determines an air quality, for example of ambient air.

The sensor is, for example, configured to detect at least one parameter of a fluid, particularly of ambient air, during operation. Here, the term “fluid” particularly means gases, liquids and/or aerosols. For example, the sensor detects humidity in the ambient air, such as a relative humidity of the ambient air and/or a dew point of the ambient air, during operation. Particularly, the sensor detects a concentration of fluids, especially of gases, such as water vapour, of a fluid mixture. For example, the sensor is a dew point sensor. For example, the sensor is configured for capacitive dew point measurement.

Documents U.S. Pat. Nos. 9,284,187 B2, 8,779,781 B2, 9,070,695 B2, 10,475,716 B2, 11,262,325 B2, US 2021/0 364 372 A1 describe sensors which are configured to be used within the sensor module. The disclosure content of each document, particularly with respect to the sensor, is incorporated herein by reference in its entirety.

According to an embodiment, the sensor module comprises a sensor cap surrounding the connection carrier and/or the sensor at least partially. Particularly, the sensor cap comprises or consists of a rigid material. For example, the sensor cap comprises or consists of a plastic, such as thermoplast or a duroplast. It is also possible that the sensor cap comprises or consists of a metal. Particularly, the sensor cap protects the connection carrier and strengthens the resistance of the sensor module to mechanical stress. Particularly, the sensor cap surrounds the sensor arranged on the connection carrier.

The sensor module is used, for example, in automotive applications. For example, the sensor module is placed at an air intake of a heating, ventilation, and air conditioning (HVAC) system and/or in the HVAC system itself to run it more power-efficiently.

According to an embodiment, the sensor module further comprises an insert surrounding the sensor, the insert leaving an access opening of the sensor freely accessible, particularly for a fluid to be detected. Particularly, the insert is an elastic and/or flexible solid body. For example, the insert has a round or circular base area seen in plan view. For example, a diameter of the insert is at least 4 millimeters seen in plan view.

According to an embodiment of the sensor module, the insert comprises or consists of an elastic material, such as a rubber. For example, the insert comprises or consists of a silicone. Particularly, the material of the insert restores its original shape after mechanical deformation, preferably completely. For example, the insert restores its original shape after mechanical deformation, preferably completely.

According to an embodiment of the sensor module, the insert is manufactured separately from the other components of the sensor module. Particularly, the insert is manufactured spatially separately from the other components and in solid form when added to the sensor module. Particularly, the insert can be removed from the sensor module in a non-destructive manner. Particularly, the insert is not a casting resin embedding the sensor and added to the sensor module in liquid form and cured to form a solid body when applied to the sensor and/or the connection carrier.

According to an embodiment of the sensor module, the insert is arranged in a first opening of the sensor cap. For example, the insert is inserted in the first opening of the sensor cap.

According to an embodiment of the sensor module, the insert has a ring-shaped base body.

According to an embodiment of the sensor module, the insert has a ring-shaped base body and a sealing lip protruding from the base body. Particularly, the sealing lip protrudes such in an inner region of the insert that the sealing lip surrounds the access opening of the sensor. Preferably, the sealing lip protects a side surface of the sensor and/or the connection carrier from ambient influences. Particularly, leads of the sensor are protected by the insert against external influences, such as moisture.

Particularly, the insert exposes only the access opening of the sensor to the ambient environment. In such a way, a dead volume of the sensor module is minimized.

According to an embodiment of the sensor module, the insert has a locking element protruding from a side face of the base body. Particularly, the locking element is attached in a mechanically stable manner to a lower face of the sensor cap. For example, the locking element is a ring-shaped protrusion surrounding the ring-shaped base body, particularly completely.

According to an embodiment of the sensor module, the sensor is arranged in an outer volume of the sensor cap and the insert closes and/or seals an inner volume of the sensor cap, particularly hermetically against fluids. For example, the connection carrier has a finger-shaped part protruding from a base part of the connection carrier. Particularly, the finger-shaped part of the connection carrier has an outer region arranged within the outer volume of the sensor cap and an inner region arranged within the inner volume of the sensor cap. The sensor is mounted to the outer region of the connection carrier. For example, at least one further electronic component of the sensor module is mounted to the inner region of the finger-shaped part and/or to the base part of the connection carrier.

Particularly, the insert presses against the connection carrier when inserted in the first opening of the sensor cap such that the inner volume of the sensor cap is sealed by the insert. Particularly, the insert protects the at least one electronic component of the sensor module arranged on the inner region of the finger-shaped part and/or on the base part of the connection carrier. For example, the electronic component is a microcontroller, particularly controlling the sensor during operation of the sensor module. Particularly, the insert protects the at least one electronic component from moisture, for example from water droplets, from the outside of the sensor module, or water that condenses when air inside the sensor module cools down below the dew point.

It is an idea of the sensor module to use the insert, which is particularly elastic and/or flexible, to protect the at least one electronic component from the ambient environment, whereas the sensor and, particularly, the access opening of the sensor is largely, preferably completely, exposed to the ambient environment, for example in order to detect ambient air.

According to an embodiment of the sensor module, the insert extends laterally over the connection carrier and engages at least partially with a penetration of the connection carrier. The penetration penetrates the connection carrier preferably completely. For example, the penetration of the connection carrier forms a planar spiral-like spring and/or a planar finger structure seen in plan view. Particularly, the sensor is arranged on or over the planar spiral-like spring and/or the planar finger structure. The finger structure or the flat spiral-like spring together with the insert provides great thermal isolation of the sensor against heat generated from the at least one electrical component on the connection carrier. Particularly, the insert presses down the planar spiral-like spring or the finger structure with the sensor such that a bottom surface of the connection carrier touches the sensor cap, and therefore superior thermal conductivity can be achieved.

According to an embodiment, the sensor module further comprises a membrane arranged over the access opening of the sensor. The membrane is in particular permeable for the fluid to be detected by the sensor. For example, the membrane is permeable for ambient air and/or water vapour. Particularly, the membrane is hydrophobic and repels water, dust and/or particles. The membrane particularly protects the sensor from harmful and/or disturbing influences from the ambient environment such as dirt, particles and/or liquid water droplets. By using the insert, it is possible to add the membrane to the sensor module in a simplified manner, particularly compared to a casting resin embedding the sensor. During a filling process of a casting resin, the membrane can be easily damaged and/or the access opening of the sensor can be covered by the casting resin.

For example, the membrane is directly attached to the sensor such that the access opening is covered by the membrane. For example, the membrane is attached to the sensor by gluing, particularly before the insert is added to the sensor module. For example, the membrane is attached to the sensor before the sensor is mounted to the connection carrier.

According to a further embodiment of the sensor module, the membrane is arranged on the sensor cap. According to a further embodiment of the sensor module, the membrane is directly attached to the sensor cap, covering the first opening of the sensor cap. For example, the membrane is attached to the sensor cap by ultrasonic welding.

According to a further embodiment of the sensor module, the sensor cap comprises a funnel-shaped recess, and the first opening of the sensor cap is comprised by a bottom face of the funnel-shaped recess. For example, the opening is surrounded by a ring-shaped area forming a support surface for the membrane. It is also possible that the first opening is arranged directly adjacent to a side face of the funnel-shaped recess. The funnel-shaped recess has, for example, a geometry of a truncated cone. Particularly, the side face of the funnel-shaped recess is a shell surface of a truncated cone.

Particularly, the bottom face of the funnel-shaped recess has a smaller diameter than an opposite second opening of the funnel-shaped recess. In such a way, the sensor has sufficient access to the ambient environment, particularly to the ambient air and/or water vapour, to be detected by the sensor.

For example, the sensor module further comprises a main housing. The main housing, for example, comprises an outer surface of the sensor module. Particularly, the main housing comprises or consists of several parts assembled with each other, for example by joining and/or by engaging.

The sensor module can be manufactured using the method described in the following. Therefore, features and embodiments disclosed in connection with the sensor module can be also embodied in the method and vice versa.

According to an embodiment of the method, a connection carrier is provided. For example, the connection carrier comprises electrical connection points and/or conductor tracks.

According to an embodiment of the method, a sensor is arranged on the connection carrier. Particularly, the sensor is mounted to the connection carrier, for example by soldering or gluing. For example, the sensor is mounted to the electrical connection point of the connection carrier in a mechanically stable and/or electrically conductive manner. Particularly, the sensor is mounted to a finger-shaped part of the connection carrier protruding from a base part of the connection carrier. Preferably, the sensor is mounted to an outer region of the finger-shaped part of the connection carrier. In other words, the sensor is mounted to a tip-part of the finger-shaped part of the connection carrier.

According to an embodiment of the method, a sensor cap is arranged over the sensor such that the sensor cap surrounds the connection carrier at least partially. Particularly, the sensor cap has a cylindrical form and surrounds the finger-shaped part of the connection carrier, for example substantially completely.

Particularly, the method comprises the following steps:
providing the connection carrier,
arranging the sensor on the connection carrier, and
arranging the sensor cap over the sensor such that the sensor cap surrounds the connection carrier at least partially.

According to an embodiment of the method, the connection carrier is arranged within a bottom part of a housing. For example, the bottom part of the housing comprises or consists of a plastic material such as a thermoplast or a duroplast. It is also possible that the bottom part of the housing comprises or consists of a metal. For example, the bottom part of the housing comprises or consists of the same material as the sensor cap.

According to an embodiment of the method, the bottom part of the housing comprises a trough-like part, in which a base part of the connection carrier is inserted. Further, the bottom part, for example, comprises a ring-shaped element at a side wall, the ring-shaped element having an outlet for the finger-shaped part of the connection carrier. For example, the finger-shaped part of the connection carrier extends laterally through the ring-shaped element of the bottom part of the housing. The ring-shaped element and the trough-like part are preferably formed as one piece.

According to an embodiment of the method, the sensor cap is mounted to the bottom part of the housing. For example, the sensor cap is mounted to the ring-shaped element of the bottom part of the housing. Preferably, the sensor cap and the bottom part of the housing are connected to each other by laser welding. This ensures water tightness and mechanical strength of the sensor module.

According to an embodiment of the method, a top cover is mounted to the bottom part of the housing. For example, the top cover is mounted to the trough-like part of the bottom part of the housing. Particularly, the top cover and the trough-like part of the bottom part form a cavity surrounding the base part of the connection carrier. In this case, the cavity is for example accessible through the ring-shaped element. Preferably, the top cover and the bottom part of the housing are connected to each other by laser welding. This ensures water tightness and mechanical strength of the sensor module.

For example, the top cover comprises a connector element. The top cover preferably further comprises ribs to improve the structural strength of the connector element.

Particularly, the bottom part, the top cover and the sensor cap form a housing of the sensor module after assembly. The bottom part, the top cover and/or the sensor cap comprise or consist of a rigid material. For example, the bottom part, the top cover and/or the sensor cap comprise or consist of the same material, such as a thermoplast, a duroplast or a metal.

According to an embodiment of the method, an insert is arranged within a first opening of the sensor cap. The insert is inserted into the first opening of the sensor cap and locks itself into place. The insert can be placed before or after assembly of the housing.

### BRIEF DESCRIPTION OF THE DRAWINGS

Further advantageous embodiments and developments of the sensor module and the method for manufacturing the sensor module will become apparent from the exemplary embodiments described below in connection with the Figures.

FIG. 1 schematically shows a perspective view of a part of a sensor module according to an exemplary embodiment;

FIG. 2 schematically shows a perspective view of a part of a sensor module according to a further exemplary embodiment;

FIG. 3 schematically shows a perspective view of a part of a sensor module according to a further exemplary embodiment;

FIGS. 4 and 5 schematically show perspective views of a part of a sensor module according to a further exemplary embodiment;

FIGS. 6 to 12 schematically show perspective views of stages of a method for manufacturing a sensor module according to an exemplary embodiment; and

FIG. 13 schematically shows a perspective view of a sensor module according to an exemplary embodiment.

Equal or similar elements as well as elements of equal function are designated with the same reference signs in the Figures. The Figures and the proportions of the elements shown in the Figures are not regarded as being shown to scale. Rather, single elements, in particular layers, can be shown exaggerated in magnitude for the sake of better presentation and/or better understanding.

### DETAILED DESCRIPTION OF ILLUSTRATIVE EMBODIMENTS

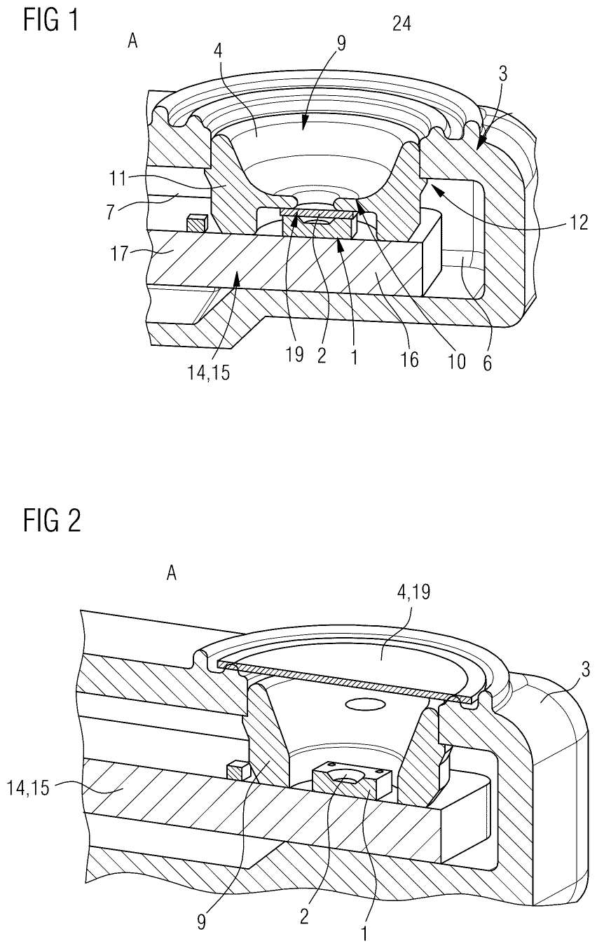

FIG. 1 shows a detail A of a sensor module according to an exemplary embodiment. For example, the detail A is marked in FIG. 13.

The sensor module according to the exemplary embodiment of FIG. 1 comprises a sensor 1, which is mounted to an outer region 16 of a finger-shaped part 15 of a connection carrier 14. The finger-shaped part 15 of the connection carrier 14 protrudes from a base part 25 of the connection carrier 14. The base part 25 of the connection carrier 14 is not shown in the detail A of FIG. 1 but is shown, for example, in FIG. 6.

The sensor 1 has an access opening 2 configured for penetration of a fluid to be detected by the sensor 1. For example, the fluid is ambient air 24 and/or water vapour.

The access opening 2 is covered with a membrane 19. The membrane 19 is permeable for the fluid to be detected, but repels dirt, particles and/or liquids being harmful for the sensor 1. At present, the membrane 19 is applied to the access opening 2 of the sensor 1 and is arranged between the sensor 1 and the insert 9.

Further, the sensor 1 comprises a sensor cap 3 surrounding the sensor 1 mounted to the connection carrier 14. Within a first opening 4 of the sensor cap 3 an insert 9 is arranged. The insert 9 extends within the sensor cap 3 and touches the connection carrier 14 in the outer region 16 of the finger-shaped part 15. Particularly, the insert 9 is a flexible solid body, which is pressed together at least slightly when mounted within the sensor cap 3, in order to seal an inner volume 7 of the sensor cap 3 to an outer volume 6 of the sensor cap 3.

The insert 9 has a ring-shaped base body 11 surrounding the sensor 1. From the ring-shaped base body 11, a ring-shaped sealing lip 10 protrudes in an inner volume of the base body 11. The sealing lip 10 covers the sensor 1 and leaves only the access opening 2 of the sensor 1 free. Particularly, the sealing lip 10 of the insert 9 lies on the sensor 1.

From a side wall of the insert 9 a locking element 12 protrudes. The locking element 12 runs in a ring-shaped manner around the side wall of the insert 9. The locking element 12 is engaged with a lower surface of the sensor cap 3, forming a mechanically stable fixation.

The sensor module according to the exemplary embodiment of FIG. 2 differs from the sensor module according to the exemplary embodiment of FIG. 1 in the location of the membrane 19. The membrane 19 of the sensor module of FIG. 2 is arranged on the first opening 4 of the sensor cap 3. Particularly, the membrane 19 covers the first opening 4 of the sensor cap 3. Further, the insert 9 is free of a sealing lip 10.

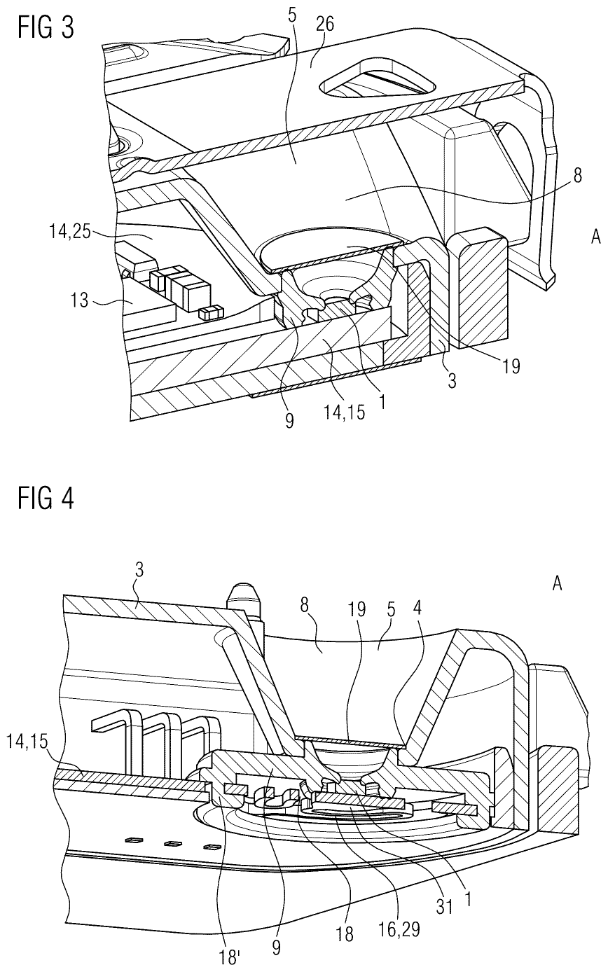

The sensor module according to the exemplary embodiment of FIG. 3 comprises a main housing 26, in which the sensor cap 3 and the connection carrier 14 with the sensor 1 mounted to the finger-shaped part 15 of the connection carrier 14 are arranged. The arrangement of the sensor cap 3, the connection carrier 14 and the sensor 1 is for example embodied as already described in connection with FIG. 2. Particularly, the sensor module of FIG. 3 comprises a membrane 19 covering a first opening 4 of the sensor cap 3 and an insert 9 surrounding the sensor 1.

Further, the connection carrier 14 comprises a base part 25 being provided with electrical components 13, for example for controlling and/or driving the sensor 1. The insert 9 seals the electrical components 13 from the ambient environment for protection.

Further, the sensor cap 3 shown in FIG. 3 comprises a funnel-shaped recess 8. A bottom face of the funnel-shaped recess comprises the first opening 4 of the sensor cap 3. A second opening 5 of the recess 8 is arranged opposite to the first opening 4 and has a larger diameter than the first opening. Particularly, the first opening 4 and the second opening 5 have a round, for example circular, shape.

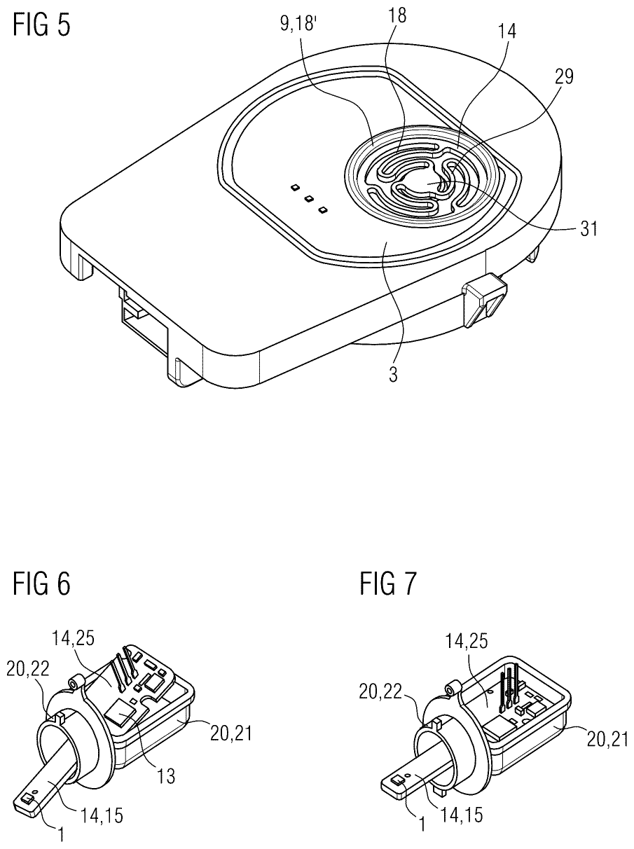

The sensor module according to the exemplary embodiment of FIGS. 4 and 5 differs in the design of the connection carrier 14 from the sensor module according to the exemplary embodiment of FIG. 3. The connection carrier 14 of the sensor module of FIGS. 4 and 5 has penetrations 18 such that a planar spiral-like spring 29 is formed within an outer region 16 of a finger-shaped part 15 of the connection carrier 14. The sensor 1 is arranged on a center part 31 of the planar spiral-like spring 29. The insert 9 extends laterally over the connection carrier 14. Particularly, an engagement part 30 of the insert 9 extends laterally over the penetrations 18 and the planar spiral-like spring 29 and engages with the outermost penetration 18′ of the planar spiral-like spring 29. The engagement part 30 of the insert 9 completely fills the outermost penetration 18′ and covers the planar spiral-like spring 29. Therefore, the insert 9 seals a volume of the sensor module from the ambient environment (see FIG. 4).

While FIG. 4 shows a perspective view of the inside of the sensor cap 3, FIG. 5 shows a perspective view on a bottom face of the sensor cap 3. As can be seen in FIG. 5, the planar spiral-like spring 29 has a round, particularly circular base area. The center part 31 of the planar spiral-like spring 29 is connected with bars to an outer ring-shaped element of the connection carrier 14. The outer ring-shaped element surrounds the center part 31.

During the method according to the exemplary embodiment of FIGS. 6 to 12, a connection carrier 14 is provided. The connection carrier 14 comprises a base part 25 and a finger-shaped part 15 protruding from the base part 25. On the finger-shaped part 15, a sensor 1 is mounted in an outer region 16. Particularly, the method according to the exemplary embodiment of FIGS. 6 to 12 is an assembly process of a sensor module.

The connection carrier 14 is inserted in a bottom part 20 of a housing (FIGS. 6 and 7). The bottom part 20 of the housing comprises a trough-like part 21, in which the base part 25 of the connection carrier 14 is placed. Further, the bottom part 20 comprises a ring-shaped element 22 arranged at a side wall of the trough-like element 21. The ring-shaped element 22 allows to push the finger-shaped part 15 of the connection carrier 14 through.

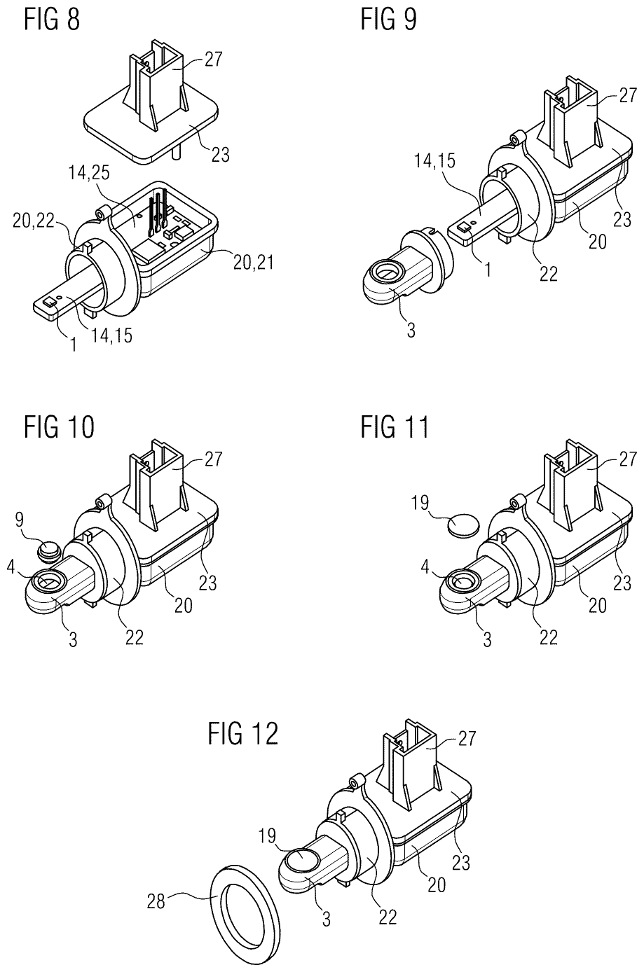

A top cover 23 of the housing is mounted to the bottom part 20 (FIG. 8), for example by laser welding. The top cover 23 and the trough-like part of the bottom part 20 form a cavity, in which the base part 25 of the connection carrier 14 is arranged. The top cover 23 further has a connector element 27.

A sensor cap 3 is mounted to the ring-shaped element 22 of the bottom part 20 (FIG. 9), for example also by laser welding.

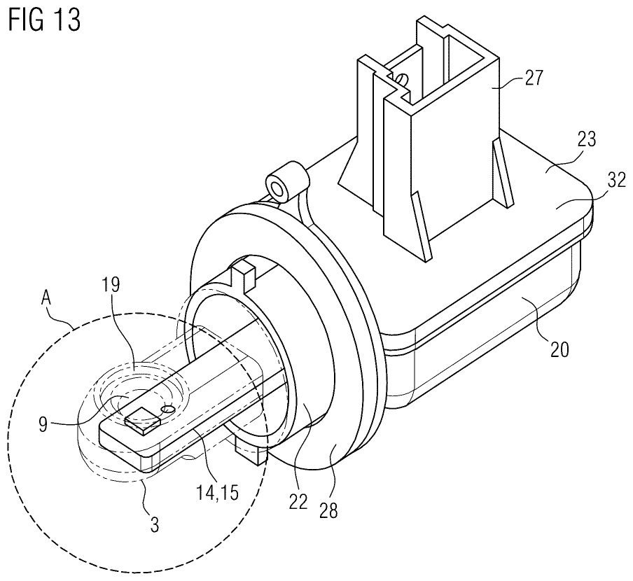

An insert 9 is placed in a first opening 4 of the sensor cap 3 (FIG. 10), and the first opening 4 of the sensor cap 3 is provided with a membrane 19 (FIG. 11), the membrane 19 completely filling the first opening 4. A sealing ring 28 is placed over the sensor cap 3 (FIG. 12).

The sensor module according to the exemplary embodiment of FIG. 13 comprises a housing 32 of a bottom part, a top cover and a sensor cap. Over a ring-shaped element of the bottom part a sealing ring is arranged. The sensor cap protrudes from the ring-shaped element. Within the sensor cap, a finger-shaped part of a connection carrier is arranged. On an outer region of the finger-shaped part of the connection carrier a sensor is applied. The detail A marked in FIG. 13 is for example embodied as described in connection with FIGS. 1 to 5.

For example, the sensor cap 3 of the sensor module is intended to be inserted in an air intake of an HVAC system and electrically connected with a connector plug via the connector element 27.

The features and exemplary embodiments described in connection with the Figures can be combined with each other according to further exemplary embodiments, even if not all combinations are explicitly described. Furthermore, the exemplary embodiments described in connection with the Figures may alternatively or additionally have further features according to the description in the general part.

The invention is not limited to the description of the exemplary embodiments. Rather, the invention comprises each new feature as well as any combination of features, particularly any combination of features of the claims, even if the feature or the combination of features itself is not explicitly given in the claims or the exemplary embodiments.

1.-14. (canceled)

15. A sensor module comprising:
a connection carrier;
a sensor arranged on the connection carrier;
a sensor cap surrounding the connection carrier and/or the sensor at least partially; and
an insert surrounding the sensor, the insert leaving an access opening of the sensor freely accessible,
wherein the insert comprises or consists of an elastic material.

16. The sensor module according to claim 15, wherein the insert is removable from the sensor module non-destructively.

17. The sensor module according to claim 15, wherein the insert is arranged in a first opening of the sensor cap.

18. The sensor module according to claim 15, wherein the insert has a ring-shaped base body.

19. The sensor module according to claim 18, wherein the insert has a locking element protruding from a side face of the base body.

20. The sensor module according to claim 15, wherein the insert has a ring-shaped base body and a sealing lip protruding from the base body.

21. The sensor module according to claim 15, wherein the insert exposes only the access opening of the sensor to an ambient environment.

22. The sensor module according to claim 15, wherein the sensor is arranged in an outer volume of the sensor cap and the insert seals an inner volume of the sensor cap.

23. The sensor module according to claim 15, wherein the insert extends laterally over the connection carrier and engages at least partially with a penetration of the connection carrier.

24. The sensor module according to claim 23, wherein the penetration of the connection carrier forms a planar spiral-like spring and/or a planar finger structure.

25. The sensor module according to claim 15, further comprising a membrane arranged over the access opening of the sensor.

26. The sensor module according to claim 25, wherein the membrane is arranged on the sensor cap.

27. The sensor module according to claim 15,
wherein the sensor cap comprises a funnel-shaped recess, and
wherein a first opening of the sensor cap comprises a bottom face of the funnel-shaped recess.

28. A method for manufacturing a sensor module, the method comprising:
providing a connection carrier;
arranging a sensor on the connection carrier;
arranging a sensor cap over the sensor such that the sensor cap surrounds the connection carrier at least partially; and
arranging an insert such that the insert surrounds the sensor, the insert leaving an access opening of the sensor freely accessible,
wherein the insert comprises or consists of an elastic material.
